(19) United States
(12) Patent Application Publication
(10) Pub. No.: US 2006/0223173 A1
(43) Pub. Date: Oct. 5, 2006

(54) Anaerobic digester for the production of methane gas from manure

(76) Inventor: Gary Nin Austin, Stillwater, OK (US)

Correspondence Address: Gary Nin Austin, 3103 N. Country Club Rd., Sillwater, OK, 74075, US

(21) Appl. No.: 11/388,430

(22) Filed: Mar. 24, 2006

Related U.S. Application Data
(60) Provisional application No. 60/667,304, filed on Apr. 1, 2005.

(51) Int. Cl. C12M 1/00

(52) U.S. Cl. 435/289.1; 435/290.1; 435/300.1

(57) ABSTRACT

An anaerobic digester to produce methane gas from animal manure is proposed to be more efficient and convenient for farmers and ranchers. The proposed invention utilizes already familiar containers such as propane tanks for the digester to produce the methane gas; and a tractor PTO (i.e.) power turn out to turn an auger within the tank. The tank is easily loaded from the top with animal manure, and then easily cleaned out with an auger turning out the resultant waste solids. Also novel to this patent application is it's simplicity and advanced enzyme catalyst.

### BACKGROUND

Anaerobic digestion of animals to produce manure and then subsequently produce methane gas has been observed for centuries. Indeed the Indians of North America observed a hotter camp-fire from using buffalo chips because of the greater BTU from the buffalo dung with a wood burning fire. During World War II Germany produced 150% of their farm energy requirements from animal manure to produce methane gas for fuel. Indeed animals that are grain fed in the stall can produce a methane gas upwards of 120 octane. Prior art has been principally pits dug to fill them with manure, and then collect the methane gas from off the top with hoses and/or tarps. Tanks that have been more effectively sealed have been shown to be too sophisticated and/or expensive for the farmer rancher to be able to afford and to use them effectively.

### SPECIFICATION

The present invention uses pressure vessels such as propane tanks and other farm and ranch components that are already familiar to the agriculture industry.

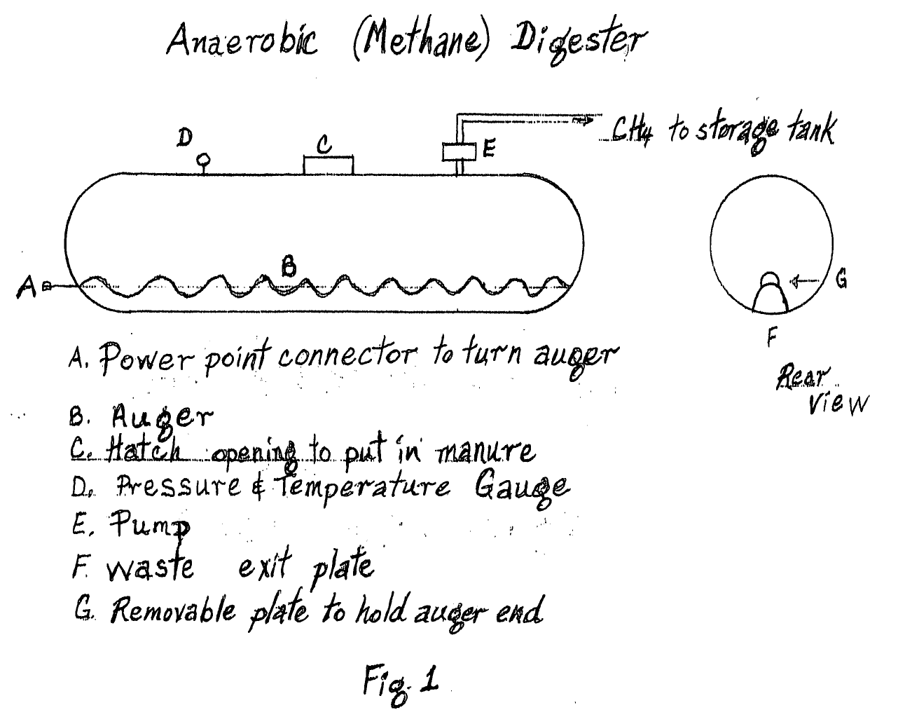

FIG. 1 is a side and rear view illustration of what is commonly seen in rural communities as a propane tank. These tanks can be easily converted to an anaerobic digester with the following modifications:

1) An opening in the top of the tank can provide a hatch that can be sealed after inserting animal manure into the tank.

2) By installing an auger in the tank that can lay on the bottom of the tank, the manure can be turned on occasion while it is being anaerobically digested.

3) The auger can be turned with an electric motor, by hand, or a tractor PTO (i.e.) power turn out.

4) A pressure and temperature gauge can be installed on top of the outside of the tank to monitor interior said conditions of the tank.

5) An electrical, mechanical or manual pump can be installed on top of the outside of the tank with or without a regulator, to pump the methane gas from the anaerobic digester to a pipeline or to an additional storage tank.

6) A removable waste exit plate at the rear of the tank can allow the interior solids remaining after digestion, to be pushed out of the digester tank with the turning of the auger inside the tank.

7) The anaerobic digester can be mounted on legs or placed on a stand, so that the waste exiting the tank can fall into a wheelbarrow, truck, or trailer.

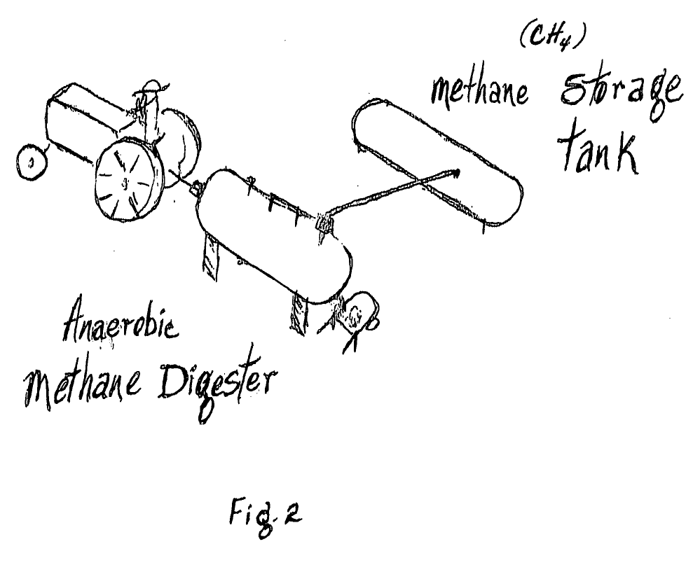

FIG. 2 Shows a tractor using it's power turn out (PTO) to turn the auger inside the anaerobic digester tank, causing the waste in the tank to exit the tank and fall into a wheelbarrow.

The methane gas generated inside the anaerobic digester is pumped into a separate storage tank.

The outside of the anaerobic digester tank is painted black to heat the interior of the tank causing the reaction to speed up, accelerating the reaction process to cook off the methane gas from the animal manure.

An enzyme catalyst can be used to catalyse a specific biochemical reaction.

An enzyme catalyst is also proposed to heat up the reaction process during cold weather that will cause the reaction to cook off the methane gas from the animal manure during colder outside temperatures.

The purpose of the enzyme catalyst being to increase the temperature of the chemical substrate—being animal manure, to produce the product methane gas (i.e.) CH₄. Wherein the enzyme catalyst is not consumed or chemically changed, but becoming denatured while causing the reaction to heat up the substrate and to produce the desired product of methane gas.

After the anaerobic digestion reaction of animal manure producing methane gas (i.e.) CH₄, the resultant catalyst along with the other bedding materials that may have been included with the manure, becomes part of the composted fertilizer that exits the anaerobic digester tank.

1) A novel anaerobic digester method utilizing tanks that are easily loaded with animal manure with a hatch that seals, and exit's the remaining solids with an auger, the purpose of which is to produce methane gas.

2) The exterior of the anaerobic digester tank is painted black to heat the interior of the vessel and speed up the anaerobic digester reaction, while being exposed outside in the sunlight.

3) The anaerobic digester can also use an enzyme catalyst to increase the temperature of the substrate (i.e.) the animal manure to speed up the reaction to produce methane gas (i.e.) CH₄ during colder climate conditions.
